(19) United States
(12) Patent Application Publication
(10) Pub. No.: US 2022/0081668 A1
(43) Pub. Date: Mar. 17, 2022

(54) IMPROVED LOOP-FERMENTER

(71) Applicant: UNIBIO A/S, Roskilde (DK)

(72) Inventor: Ib Christensen, Allerod (DK)

(21) Appl. No.: 17/420,418

(22) PCT Filed: Jan. 24, 2020
(86) PCT No.: PCT/EP2020/051787

§ 371 Date: Jul. 2, 2021

(30) Foreign Application Priority Data
Jan. 25, 2019 (DK) PA 2019 00106

(51) Int. Cl. C12M 1/00; C12M 1/21; C12M 1/02

(52) U.S. Cl. CPC C12M 29/18; C12M 29/20; C12M 23/22; C12M 27/00; C12M 41/02

(57) ABSTRACT

A fermentation reactor is shown with a loop-part and a top tank, the loop-part having a downflow part, connected to an upflow part via a U-part, wherein the top tank includes: (i) a first outlet connecting the top tank to the downflow part of the loop-part and allowing a fermentation liquid present in the top tank to flow from the top tank into the loop-part; (ii) a first inlet connecting the top tank to the upflow part of the loop-part, allowing fermentation liquid present in the loop-part to flow from the loop part into the top tank; and (iii) a vent tube for discharging effluent gasses from the top tank; wherein the top tank further includes a visual inspection means.

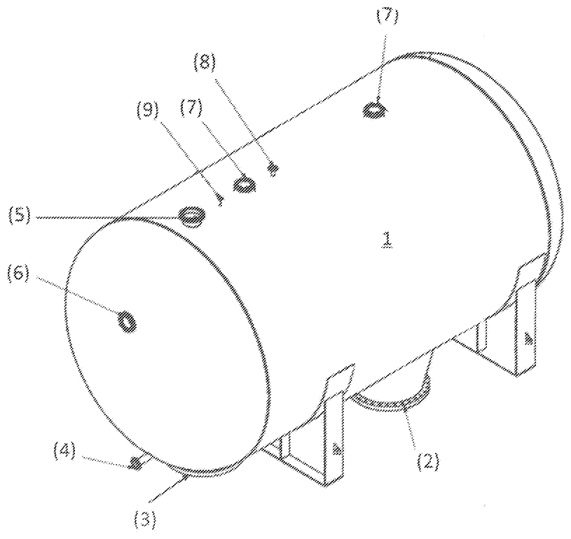

### TECHNICAL FIELD OF THE INVENTION

The present invention relates to an improved fermentation reactor. In particular, the present invention relates to an improved fermentation reactor comprising a loop-part and a top tank.

### BACKGROUND OF THE INVENTION

When fermenting methanotrophic organisms the degassing in the top tank is of outmost importance as insufficient degassing of the fermentation liquid to liberate waste gasses, like CO2, which in certain amounts may accumulate and become toxic to the methanotrophic organisms fermenting in the fermentation reactor.

During fermentation using a fermentation reactor comprising a loop-part and a top tank, also called a Loop-reactor, the fermentation liquid is circulated from the top tank into the downflow part through the U-part into the upflow part and returning to the top tank. When the fermentation liquid enters the top tank, foam is developed due to the headspace available in the top tank.

It is important to control the foam development during fermentation, as too much foam may constitute a risk of the fermentation process, where too extensive foaming may increase the risk of explosions as the gas in the foam mainly may constitute methane and when released the concentration of methane may significantly increase and create a ratio with oxygen within the explosive area.

In the event excessive amounts of foam are generated, an antifoaming agent are used which may have the consequence of setting the fermentation process on hold and there is a risk the fermentation process may be restarted.

In order to control extensive foaming, electronical foam detectors or sensors are traditionally used. However, the challenge of using electronic foam detectors/sensors is that when activated the fermentation process is stopped and occasionally the fermentation process must be started all over again. Another possibility may be adding anti-foaming to the fermentation liquid to reduce the foam development, as described above with the means for “treating” excessive foam development. As mentioned previously, the disadvantages of using anti-foaming agents is that the gas responsible for building up the foam mainly constitute methane and when liberated the methane concentration in the top tank is instantly and significantly increased. When the foam level is high the concentration of methane becomes even further increased and a ratio between methane and oxygen may be within the explosive area, which should be avoided. Furthermore, when foaming agents are added to the fermentation liquid, degassing may be worsened, and addition of methane must be stopped, and the cells may be periodically starved.

Hence, an improved fermentation reactor would be advantageous, and in particular a more efficient and/or reliable fermentation reactor where it is possible to control both insufficient foaming and too extensive foaming, where degassing may be improved, where delays are reduced or avoided and costs may be reduced when fermenting methanotrophic organisms would be advantageous.

### SUMMARY OF THE INVENTION

Thus, an object of the present invention relates to an improved fermentation reactor.

In particular, it is an object of the present invention to provide a fermentation reactor that solves the above-mentioned problems of the prior art with degassing and at the same time avoid too extensive foaming, reducing the risk of explosions, the risk of the fermentation process may stop and occasionally must be started all over again.

Thus, one aspect of the invention relates to a fermentation reactor comprising a loop-part and a top tank, said loop-part comprising a downflow part, connected to an upflow part via a U-part, wherein the top tank comprises:
(i) a first outlet connecting the top tank to the downflow part of the loop-part and allowing a fermentation liquid present in the top tank to flow from the top tank into the loop-part;
(ii) a first inlet connecting the top tank to the upflow part of the loop-part, allowing fermentation liquid present in the loop-part to flow from the loop part into the top tank; and
(iii) a vent tube for discharging effluent gasses from the top tank;
wherein the top tank further comprises a visual inspection means.

Another aspect of the present invention relates to a process of fermenting at least one methanotrophic organism, or a co-fermentation comprising at least one methanotrophic organism, wherein the process comprises the step of:
(a) adding at least one methanotrophic organism; necessary substrates, such as nutrient salts, pH adjusting components and water; and at least one gaseous substrate component

into a fermentation reactor comprising a loop-part and a top tank, said loop-part comprising a downflow part, connected to an upflow part via a U-part, wherein the top tank comprises:
(i) a first outlet connecting the top tank to the downflow part of the loop-part and allowing a fermentation liquid present in the top tank to flow from the top tank into the loop-part;
(ii) a first inlet connecting the top tank to the upflow part of the loop-part, allowing fermentation liquid present in the loop-part to flow from the loop part into the top tank; and
(iii) a vent tube for discharging effluent gasses from the top tank;
wherein
the top tank further comprises a visual inspection means.

Yet another aspect of the present invention relates to the use of a fermentation reactor comprising a loop-part and a top tank, said loop-part comprising a downflow part, connected to an upflow part via a U-part, wherein the top tank comprises:
(i) a first outlet connecting the top tank to the downflow part of the loop-part and allowing a fermentation liquid present in the top tank to flow from the top tank into the loop-part;
(ii) a first inlet connecting the top tank to the upflow part of the loop-part, allowing fermentation liquid present in the loop-part to flow from the loop part into the top tank; and
(iii) a vent tube for discharging effluent gasses from the top tank;
wherein
the top tank further comprises a visual inspection means;

for fermenting at least one methanotrophic organism.

### BRIEF DESCRIPTION OF THE FIGURES

The sole FIGURE shows a top tank (1) of a fermentation reactor for fermenting at least one methanotrophic comprising a loop-part and a top tank (1). The loop-part (not shown in the FIGURE) comprising a downflow part (not shown), connected to an upflow part (not shown) via a U-part (not shown), wherein the top tank (1) comprises: (i) a first outlet (2) connecting the top tank (1) to the downflow part of the loop-part and allowing a fermentation liquid present in the top tank (1) to flow from the top tank (1) into the loop-part; (ii) a first inlet (3) connecting the top tank (1) to the upflow part of the loop-part, allowing fermentation liquid present in the loop-part to flow from the loop part into the top tank (1); and (iii) a vent tube (5) for discharging effluent gasses from the top tank. In the illustration shown in the FIGURE, the top tank (1) has been provided with a product outlet (4) for obtaining the biomass (the fermentation product). In another embodiment the product outlet may be placed in the loop-part of the fermentation reactor, and preferably in the U-part of the loop-part. The top tank (1) is further provided with a visual inspection means (6), in particular the visual inspection means (6) may be provide as an inspection hole, or as a sight glass (6), optionally in combination with a camera. In addition to the visual inspection means (6) the top tank (1) may be provided with at least one foam sensor (8) inside the top tank. In case the level of foam is increased, one way to reduced the level of foam may be to add a defoaming agent through a defoaming inlet (9). In order to improve the visual inspection, the top tank (1) may be provided with a light source (7). The light source may be provided as an individual feature or as an integrated feature in the sight glass (6).

The present invention will now be described in more detail in the following.

### DETAILED DESCRIPTION OF THE INVENTION

Accordingly, the inventor of the present invention surprisingly found that controlling foaming and turbulence of the fermentation liquid in the top tank of a U-loop reactor had significant influence on the degassing of effluent gasses.

Hence, ensuring sufficient foaming as well as sufficient turbulence in the fermentation liquid resulted in an improved productivity of the fermentation process. Thus, there is an interest in providing a stable degree of foaming of the fermentation liquid.

Thus, a preferred aspect of the present invention relates to a fermentation reactor comprising a loop-part and a top tank, said loop-part comprising a downflow part, connected to an upflow part via a U-part, wherein the top tank comprises:
(i) a first outlet connecting the top tank to the downflow part of the loop-part and allowing a fermentation liquid present in the top tank to flow from the top tank into the loop-part;
(ii) a first inlet connecting the top tank to the upflow part of the loop-part, allowing fermentation liquid present in the loop-part to flow from the loop part into the top tank; and
(iii) a vent tube for discharging effluent gasses from the top tank;

wherein
the top tank further comprises a visual inspection means.

The U-part of the loop-reactor may be connecting the lower part of the downflow part to the lower part of the upflow part. Furthermore, the upper part of the upflow part may be connected to the first inlet connecting the top tank to the upper part of the upflow part. The first outlet may be connecting the top tank to the upper part of the downflow part

In the present context the term “fermentation reactor” relates to a reactor comprising a top tank connected to the upper ends of a downflow part and an upflow part. The downflow part and the upflow part are connected at the lower ends via a U-part.

Conventional fermenters are stirred tanks which may be provided with or without recirculation systems or sampling conduits. In these types of fermenters or reactors mixing of gases with the fermentation liquid is effected by means of stirrer blades placed centrally in the fermenter and gaseous substrates is added to the stirred tank. The stirrer blades generate turbulence in the liquid, which means that gas, usually injected at the bottom of the reactor, will be dissipated in the liquid in the form of small fine gas bubbles.

The gaseous substrates are added at the bottom of the stirred tank and must be pressurized to overcome the hydrostatic pressure in the tank into which they are pumped. This compression of gases requires significant amounts of energy.

This type of reactor provides a relatively homogenous mixing, i.e. that about the same concentrations of gases and substrates will be found whether measuring at the top or at the bottom of the reactor. But the vigorous mixing in order to create small gas bubbles and ensure optimal mixing in the tank also requires the use of excessive energy and further implies a significant heating of the fermentation liquid. The excessive use of energy renders this type of reactor uneconomical, especially for cheap products such as microbial cells, which are currently sold as animal food or fish food.

Other fermenter types have also been designed with the intention of reducing energy consumption for mixing but still ensuring sufficient mass transfer of gases to the liquid phase. These fermenters are often called air lift fermenters, jet loop fermenters or U-loop fermenters.

Different types of air lift reactors have been designed in order to avoid the mechanical stirring. The majority of these reactors are so-called loop reactors having two sections: an up-flow part and a down-flow part, which are interconnected at both ends. Gases are supplied as small bubbles at the bottom of the reactor in the up-flow part usually in a nozzle arrangement. The bubbles mix with the liquid, whereby the total density is reduced and the gas-liquid mixture ascends while being displaced by new liquid emerging from the down-flow part. The gas-liquid mixture moves up through the up-flow part of the reactor and releases gas bubbles at the top. Then, the liquid descends down through the down-flow part. In order to obtain a long residence time for the gas bubbles in the liquid. Airlift reactors are conventionally very tall slender reactors, and the gas must be supplied at a high pressure for overcoming the hydrostatic pressure at the bottom of the reactor. If the gas is air, this implies the use of compressors. Compression of air usually requires significant amounts of energy.

Airlift reactors have a relatively poor exploitation of the injected gas. Typically, only 20-40% of the oxygen gas is utilized. It is often difficult to obtain a good and quick release of the gas bubbles from the fermentation liquid at the top of the reactor and separation of the gas phase thus produced (which may be rather foaming) from the liquid phase before the liquid flows into the down-flow part of the reactor. The gas phase, including significant amounts of waste gases from the fermentation, e.g. CO₂, is thus entrained in the broth, is then re-dispersed in the broth, which may lead to a reduced solubilization of the substrate gases added to the fermenter.

The U-loop reactor as described in the present invention has a simple design and is constructed with a view to provide non-compressed or nearly non-compressed substrate gas injection in the U-part in combination with a long residence time for the gases throughout the U-part and thus a high degree of exploitation of the injected gases. The top of the reactor comprises a top tank which is designed to achieve a good separation of gases and liquid.

In the present context the term “loop reactor” relates to a specific example of a fermentation reactor. The loop reactor according to the present invention comprises a top tank and a loop-part. Preferably, the loop reactor according to the present invention may be a U-loop reactor.

The “loop reactor” according to the present invention may preferably be defined by having a length of the loop-part which may be longer than the length and/or the height of the top tank. Preferably, the loop reactor or the fermentation reactor according to the present invention may have a loop part having a length which is longer, preferably substantially longer, than the length and/or the height of the top tank.

In an embodiment of the present invention the fermentation reactor according to the present invention may have a loop part having a length which is longer, preferably substantially longer, than the length and/or the height of the top tank.

In a further embodiment of the present invention, the length of the loop-part is at least 125% (v/v) longer than the length and/or the height of the top tank; such as at least 150% (v/v); e.g. at least 200% (v/v); such as at least 300% (v/v); e.g. at least 400% (v/v); such as at least 600% (v/v); e.g. at least 800% (v/v); such as at least 1000% (v/v); e.g. at least 1500% (v/v); such as at least 2000% (v/v); e.g. in the range of 125-2000% (v/v); such as in the range of 150-1500% (v/v); e.g. in the range of 200-1000% (v/v); such as in the range of 300-800% (v/v); e.g. in the range of 400-600% (v/v).

In yet an embodiment of the present invention the fermentation reactor according to the present invention may have a loop part having a length which may be longer, preferably substantially longer, than the length and/or the height of the top tank; and a volume of the top tank being larger than the volume of the loop-part.

The loop part of the present invention relates to the downflow part, the upflow part as well as the connecting part at the lower ends of the upflow part and the downflow part formed by a U-part. Hence, the “loop part” relates to the fermentation reactor, without the top tank.

In the present context the term “U-part” relates to bend provided in the bottom part of the fermentation reactor or the loop reactor connecting the lower ends of the upflow part and the downflow part. Preferably, the upflow part and the downflow part are vertical or substantially vertical.

The fermentation reactor according to the present invention comprises a loop-part and a top tank, said loop-part comprising a downflow part, connected to an upflow part via a U-part. The U-part according to the present invention may have a substantially horizontal connecting part, which connects the lower end of the down-flow part with the lower end of the up-flow part.

In a preferred embodiment of the present invention, the fermentation reactor and/or the loop-reactor according to the present invention may be a U-loop reactor.

In a further embodiment of the present invention the downflow part and the upflow part has substantially the same vertical lengths. In the present context the term “vertical length” relates to a downflow part or an upflow part wherein the vertical upflow part may be a single downflow part or a single upflow part or it may be divided into two or more vertical downflow parts or two or more vertical upflow parts which in combination (combination of the two or more vertical downflow parts or the combination of the two or more vertical upflow parts) represents the vertical downflow part or the vertical upflow part.

The length of the horizontal connecting part may vary depending on the type of loop reactor to be provided and/or used.

Fermentation reactor may be designed as a vertical loop fermenter or a horizontal loop fermenter.

In an embodiment of the present invention the fermentation reactor may be a vertical loop reactor. A vertical loop reactor may relate to a loop reactor having a main part of the U-part in vertical, or substantially vertical, position relative to horizontal position. In an embodiment of the present invention the fermentation reactor comprises a main part of the U-part in vertical, or substantially vertical, position.

In another embodiment of the present invention the fermentation reactor may be a horizontal loop reactor. A horizontal loop reactor may relate to a loop reactor having a main part of the U-part in horizontal, or substantially horizontal, position relative to vertical position. In an embodiment of the present invention the fermentation reactor comprises a main part of the U-part in horizontal, or substantially horizontal, position.

Preferably, the fermentation reactor may be designed as a vertical loop fermenter.

In the context of the present invention the term “main part” relates to at least 51% (v/v) of the U-part having the desired position; such as at least 55% (v/v); e.g. at least 60% (v/v); such as at least 65% (v/v); e.g. at least 70% (v/v); such as at least 75% (v/v); e.g. at least 80% (v/v); such as at least 85% (v/v); e.g. at least 90% (v/v); such as at least 95% (v/v); e.g. at least 98% (v/v).

The U-part of the fermentation reactor of the present invention may be designed in different ways.

In one embodiment of the present invention the loop-part may be designed with a single down-flow part and a single up-flow part connected by a U-part.

In another embodiment of the present invention the down-flow part and/or the up-flow-part may comprise two or more down-flow part and/or up-flow-part regions. Examples of two or more down-flow part and/or up-flow-part regions may be found in WO 2018/132379 which is hereby incorporated by reference.

In the present context the term “top tank” relates to a container located at the top of the fermentation reactor and responsible for removal of effluent gas from the fermentation liquid. Preferably, the top tank is during operation/fermentation only partly filled with fermentation liquid. In an embodiment of the present invention the term “partly filled with fermentation liquid” relates to a 90:10 ratio between fermentation liquid and gas; such as an 80:20 ratio; e.g. an 70:30 ratio; such as an 60:40 ratio; e.g. an 50:50; such as an 40:60 ratio; e.g. an 30:70 ratio; such as an 20:80 ratio; e.g. an 10:90 ratio.

In the context of the present invention the “visual inspection means” relates to one or more means allowing the skilled person to obtain direct information on the foaming characteristics in the top tank.

In an embodiment of the present invention the direct information may be real time information on the foaming characteristics in the top tank.

In a further embodiment of the present invention the foaming characteristics in the top tank may involve, foaming density, foaming height, and level of turbulence provided in the top tank.

The turbulence in the top tank may be provided in the fermentation liquid present in the top tank when the fermentation liquid is forced from the upflow part through the first inlet and into the top tank.

The foaming density may be an expression of the size of the bubbles in the foam. The larger the bubbles in the foam the smaller the foaming density, smaller kg foam/m³. The smaller the bubbles in the foam the larger the foaming density, larger kg foam/m³.

In an embodiment of the present invention the visual inspection means may be placed with a horizontal or substantial horizontal inspection view.

In a further embodiment of the present invention the visual inspection means may be placed on the side of the top tank allowing a combined view above the surface of a fermentation liquid and below the surface of the fermentation liquid.

Preferably, the visual inspection means may be placed in the end of the top tank.

Even more preferably, the visual inspection means may be placed in the end of the top tank providing a view from the first inlet (or the upflow part) towards the first outlet (or the downflow part).

In an embodiment of the present invention the visual inspection means may be an inspection hole, a camera, or a combination of an inspection hole and a camera.

Preferably, the inspection hole may be a sight glass.

The camera may be an inline camera.

In an embodiment of the present invention the top tank may be provided with a light source in order improve the visual inspection inside the top tank. The light source may be provided as a window allowing surrounding light to enter the top tank and/or as an artificial light source incorporated into the top tank.

In a further embodiment of the present invention, the light source may be provided as an individual feature (e.g. as an individual artificial light source) or as an integrated feature (e.g. as an integrated artificial light source) in the sight glass.

In addition to the visual inspection means the top tank may be provided with at least one foam sensor inside the top tank.

In order to avoid and/or handle excessive foam development a defoaming agent may be added to the fermentation liquid. Thus, the top tank may be provided with a defoaming inlet.

The fermentation reactor may be provided with one or more sensors to control the level and/or the addition of gaseous substrates, water, minerals nutrition etc.

In an embodiment of the present invention the fermentation reactor, preferably the loop-part comprises an ion sensor or analyzer for determining the content of one or more ion species in a fermentation liquid, preferably, the one or more ion species is selected from phosphate, calcium, hydrogen, nitrate, nitrite and/or ammonium, preferably nitrate and/or nitrite.

In a further embodiment of the present invention the loop reactor may be provided with a circulation pump. The circulation pump may preferably be placed in the loop-part of the loop reactor.

Preferably, the circulation pump may be placed in the upper half part of the downflow part.

In an embodiment of the present invention, the fermentation reactor may comprise a flow reducing device. Preferably, the flow reducing device may be inserted upstream from the first inlet and in the upper half of the upflow part.

In a further embodiment of the present invention the loop-part of the fermentation reactor may preferably comprise one or more gas inlets; one or more water inlets; and/or one or more fermentation medium inlets.

The one or more gas inlets; the one or more water inlets; and/or the one or more fermentation medium inlets may be controlled by a computer. Preferably, the one or more gas inlets; the one or more water inlets; and/or the one or more fermentation medium inlets may be controlled by a computer based on the data obtained from the one or more sensors or analyzers.

In order to provide improved fermentation conditions distribution of gaseous substrate, such as methane in the fermentation liquid may be important. Thus, the loop-part of the fermentation reactor may comprise one or more active devices for distributing gas in the fermentation liquid

In an embodiment of the present invention the one or more active devices for distributing gas in the fermentation liquid is a micro- or nano-sparger for introducing and/or distributing gas into the fermentation liquid; and/or a dynamic motion device placed in the loop part of the reactor, such as a dynamic mixer.

In addition to, or as an alternative to, the dynamic mixers, the loop-part may comprise one or more inactive mixing members. In an embodiment of the present invention the one or more inactive mixing members may be a static mixer.

In an embodiment of the present invention the loop reactor may comprises at least one static mixer, at least one dynamic mixer or comprises at least one static mixer and at least one dynamic mixer.

In addition to the importance of proper degassing in the top tank, it may be important to improve the mass transfer of the gaseous substrates into the liquid phase where the gas becomes available to the biocatalysts (e.g. the methanotrophic organisms) in an energy efficient manner.

Furthermore, as mentioned it may also be important to improve the efficiency of the waste gas removal by improving waste gas transfer from the liquid phase into the gas phase for removal from the fermenter, preferably done in the top tank.

Preferably, this improved efficiency in waste gas removal may be provided by operating the U-part of the loop part under increased pressure and having atmospheric pressure, or even vacuum, in the top tank.

This improved mass transfer in combination with improved gas removal in the top tank may be achieved with the fermentation reactor, the loop reactor, according to the invention, which comprises a loop-part having an essentially vertical down-flow part, an essentially vertical up-flow part and a U-part having a substantially horizontal connecting part, which connects the lower end of the down-flow part with the lower end of the up-flow part, a top tank which may be provided above the loop-part and connects the upper end of the down-flow part and the upper end of the up-flow part.

In an embodiment of the present invention the top tank may have a diameter which is substantially larger than the diameter of any one or more of the loop-part, the down-flow part, and the up-flow part.

In a further embodiment of the present invention the volume of the loop-part is larger, preferably, substantially larger, than the volume of the top tank.

In yet another embodiment of the present invention the top tank may have a diameter which is substantially larger than the diameter of loop-part, the down-flow part, and/or the up-flow part and the length of the loop-part may be larger, preferably, substantially larger, than the length or height of the top tank.

The fermentation reactor may comprise a liquid circulation means, preferably in form of a circulation pump.

In an embodiment of the present invention the fermentation reactor may comprise an outlet, preferably the outlet may be placed in the top tank or in the U-part of the loop part of the fermentation reactor, for withdrawing fermentation liquid.

The fermentation reactor may comprise one or more gas injection points, which, according to wishes and demands, are placed in the down-flow part, the U-part and/or the up-flow part. Preferably, the one or more gas injection points are placed in the down-flow part.

Directly following the one or more gas injection points, at least one active mixing member and/or at least one inactive mixing member is provided for dispersion of the gas(ses) introduced into the fermentation liquid.

It has been demonstrated that by increasing the pressure in the U-loop, loop reactor, an increased mass transfer from the gaseous phase to the liquid phase may be provided. Thus, a first pressure controlling device may be inserted in the U-part of the fermenter for increasing the pressure in at least a first zone of the U-part in the fermenter in relation to the pressure in a second zone of the fermenter.

In a preferred embodiment of the present invention the first pressure controlling device may be inserted in the upper end of the down-flow part, and a second pressure controlling device may be inserted in the U-part of the fermenter and downstream of the first pressure controlling device when seen in the flow direction of the fermentation liquid.

The first pressure controlling device may be a valve (e.g. commercially available valve types), a pump, e.g. a propeller pump, a lobe pump, or a turbine pump, or the pressure may be increased by injection of pressurized air or another gas, e.g. an inert gas. The first pressure controlling device is preferably a propeller pump, which also creates liquid circulation in the fermenter.

The second and optionally a third pressure controlling device may be placed in the down-flow part, the up-flow part, or in the U-part, but preferably the second pressure controlling device is in the upper half part of the up-flow part. The third optional pressure controlling device is preferably placed in the upper half part of the up-flow part and downstream to the second pressure controlling device when seen in the flow direction of the fermentation liquid. The second and/or third pressure controlling devices are chosen among a group of devices comprising a valve (e.g. commercially available valve types), a static mixer, a hydrocyclone, a pump (e.g. a propeller pump, a lobe pump or a turbine pump), a pressure controlled valve, a plate with holes, nozzles or jets or a narrowing of the diameter or cross-section of the fermenter part in which it is placed.

In an embodiment of the present invention an improved mass transfer of the gaseous substrate may be provided in the U-part of the fermentation reactor according to the present invention.

In a further embodiment of the present invention the waste gas removal may be provided in the top tank of the fermentation reactor according to the present invention.

In an embodiment of the present invention means are provided in order to permit flushing of the headspace to improve waste gas removal and reduce the risk of explosive gas mixtures being formed in the headspace of the fermenter.

This flushing may be achieved by placing gas flushing means in the top tank, such as devices for adding and/or removing a gas in a headspace. The gas flushing means may preferably be placed above the liquid surface for creating a gas flow of flushing gas co-currently, con-currently or cross-currently to the liquid flow in the top part of the fermenter. The gas adding means may also be placed below the liquid surface in the top part. Alternatively, or additionally, waste gas removal may be increased by reducing the pressure in the headspace by applying suction or a vacuum, thus reducing the pressure in the headspace and/or by installing flow modifying means in the top part. The invention also permits the energy applied to increase the pressure to be recovered for reuse. This may be achieved by connecting the second, and optionally the third pressure controlling device to a brake or a generator for decreasing the pressure with the propeller pump. If a generator is connected to the second and/or third pressure controlling device, some of the energy applied to the system may be collected, thus reducing the overall energy consumption of the system.

In the present context the term “flushing” is used in respect of a process performed in the top tank for removing or assisting removal of effluent gas from the head space of the top tank and/or from the fermentation liquid in the top tank.

The top tank provided according to the present invention may be designed to contain between 1% and 99% of the overall volume of the fermenter, but preferably between 10% and 60% of the overall fermenter volume, even more preferably between 40-50% of the overall fermentation volume. In an embodiment of the present invention the volume of the top tank may be less than the volume of the U-part.

The top tank may be provided with liquid or gas flow modifying means in order to assist mixing in the fermentation reactor or to assist gas bubble release from the fermentation liquid. The gas or liquid flow modifying means may be dynamic mixers, baffles or static mixers.

The size, i.e. both the diameter, the length, and/or the height of the loop-part and the size of the top tank may vary according to the needs of total fermenter volume.

In an embodiment of the present invention the fermentation reactor according to the present invention may be provided with driving gas inlet where a driving gas may be introduced to drive carbon dioxide in the liquid phase into a separable effluent gas phase. The driving gas inlet may preferably be placed upstream from the top tank and/or upstream from the first inlet.

The driving gas, i.e. the gas used to displace carbon dioxide from the dissolved phase (usually nitrogen but optionally another inert non-flammable gas) may for example be introduced at one or more points from the beginning of the substantially vertical up-flow zone to the entry into the effluent gas removal zone, however particularly preferably it will be introduced at one or more points between the upper portion (e.g. the upper 20%, more preferably the upper 10%) of the vertical portion of the up-flow zone and the beginning of the flattest (i.e. most horizontal) portion of the out-flow zone.

In the context of the present invention the term “driving gas” is used in respect of a process performed in loop part, preferably in the upper end of the upflow part, and is assisting removal of effluent gas from the fermentation liquid into the gaseous phase.

In an embodiment of the present invention the fermentation reactor includes both an inlet in the top tank for introducing a flushing gas into the top tank and an inlet in the upper end of the upflow part of the loop part for introducing a driving gas for moving effluent gas from the fermentation liquid into the gaseous phase.

One advantages of the present invention that may be that an improved utilization of the gaseous substances added to the fermentation reactor may be provided Therefore, the invention may also relate to a fermentation reactor and a fermentation method of performing a fermentation process, in which at least one of the substrates may be a gas.

The fermentation method may comprise the steps of adding fermenting microorganisms, necessary substrates, such as nutrient salts, pH adjusting components and water, and at least one gaseous substrate component, such as methane, into the loop reactor, and fermenting while the fermentation liquid is circulated in the loop reactor by liquid circulating means, withdrawing a product stream from the fermenter and optionally recycling recovered fermentation liquid (supernatant), if any, to the loop reactor.

The loop reactor and the method of fermenting microorganisms according to the present invention may comprise means for controlling the pressure in the loop reactor.

The fermentation method according to the present invention may comprise the steps of controlling the pressure differently in the circulating fermentation liquid in at least two different zones in the loop reactor by increasing the pressure in at least a first zone of the U-part or the loop part of the fermentation reactor in relation to the pressure in another zone of the fermentation reactor, thereby increasing the mass transfer of the at least one added gaseous substrate component, such as methane, from the gas phase into the liquid phase in that zone, followed by decreasing the pressure in another zone in relation to the pressure in the first zone, before the circulating fermentation liquid enters the top part of the reactor, which initiates liberation of gases, such as effluent gases like CO₂, from the liquid phase, and releasing gases trapped in the circulating fermentation liquid in the top tank of the fermentation reactor.

The productivity of the fermentation reactor and/or the fermentation process according to the present invention may be further optimized in that the circulating fermentation liquid experiences an alternating pressure during circulation in the fermenter and has an increased mass transfer and solubility of substrate gases into the liquid phase in the zone having an increased pressure. The productivity may also be improved by release of gases, such as waste gases from the circulating fermentation liquid, which release is increased in the zones where the pressure is reduced.

The fermentation reactor and/or the fermentation method according to the present invention may be further improved by creating a third zone between the second pressure controlling device and a third pressure controlling device and controlling the pressure differently in the circulating fermentation liquid in each of the three different zones, the first zone, the second zone and the third zone. This control of the pressuring in the third zone may be provided by increasing the pressure in the first zone by the first pressure controlling device, and decreasing the pressure in the subsequent two zones (in the second zone and in the third zone) in two steps by the second and third pressure controlling devices.

In an embodiment of the present invention the increased pressure in the loop part of the fermentation reactor, in the first zone and/or between the first pressure controlling device and the second pressure controlling device may be provided by applying a pressure above 0.5 bar above atmospheric pressure; such as a pressure above 1 bar above atmospheric pressure; e.g. a pressure above 1.5 bar above atmospheric pressure; such as a pressure above 2 bar above atmospheric pressure; e.g. a pressure above 2.5 bar above atmospheric pressure; such as a pressure above 3 bar above atmospheric pressure; e.g. a pressure above 3.5 bar above atmospheric pressure; such as a pressure above 4 bar above atmospheric pressure; e.g. a pressure above 4.5 bar above atmospheric pressure; such as a pressure above 5 bar above atmospheric pressure; e.g. a pressure above 5.5 bar above atmospheric pressure such as a pressure above 6 bar above atmospheric pressure; e.g. a pressure above 7 bar above atmospheric pressure.

In another embodiment of the present invention the increased pressure in the loop part of the fermentation reactor, in the first zone and/or between the first pressure controlling device and the second pressure controlling device may be provided by applying a pressure in the range of 0.5-bar above atmospheric pressure; such as a pressure in the range of 1-9 bar above atmospheric pressure; e.g. a pressure above 1.5-8 bar above atmospheric pressure; such as a pressure in the range of 2-7 bar above atmospheric pressure; e.g. a pressure above 3-6 bar above atmospheric pressure; such as a pressure in the range of 4-5 bar above atmospheric pressure.

In an even further embodiment of the present invention the pressure in the top tank may be less than 0.5 bar above atmospheric pressure; such as 0.25 bar above atmospheric pressure; such as 0.1 bar above atmospheric pressure; such as about atmospheric pressure; e.g. below 0.75 bar below atmospheric pressure; such as 0.5 bar below atmospheric pressure; e.g. below 0.25 bar below atmospheric pressure; such as 0.1 bar below atmospheric pressure.

The first pressure controlling device may preferably be a pump, e.g. a propeller pump, a lobe pump or a turbine pump especially designed for circulating a gas-liquid mixture. Other suitable means for increasing the pressure and creating liquid circulation in the fermenter are e.g. addition of a pressurized gas, e.g. air or an inert gas in combination with a liquid circulating device, which may be a pump.

The second pressure controlling device according to the present invention may be chosen among a number of pressure controlling devices such as: a narrowing of the diameter/cross section of the upflow part or of the U-part, a plate with holes, jets or nozzles inserted in the upflow part or the U-part, a valve, e.g. a valve controlled by the pressure at one or more locations in the fermenter, a static mixer, a hydro cyclone or a pump, such as a propeller pump, a lobe pump or a turbine pump.

As mentioned previously, gas separation of waste gases in the fermenter may be improved by adding means for flushing the headspace and/or the fermentation liquid in the top tank. This may be achieved by creating a gas flow of flushing gas for flushing the headspace co-currently, con-currently or cross-currently to the liquid flow in the top part. Gas separation may also be further improved by adding the flushing gas (e.g. air, CO₂ or an inert gas or mixtures thereof) in the top tank below the liquid surface for increasing stripping of gases from the fermentation liquid in the top tank and into the headspace. This may also be achieved by the fermentation liquid passing flow modifying means in the top tank.

In an embodiment of the present invention the fermentation reactor and the method according to the present invention is for methanotrophic fermentation.

In a further embodiment of the present invention the fermentation reactor may be for the fermentation of methanotrophic organisms.

In an embodiment of the present invention, the gaseous substrate, to be supplied during the fermentation of methanotrophic organisms, may comprise an alkane.

Preferably, the alkane is a Cl compound. In a preferred embodiment of the present invention the alkane may preferably be a Cl compound and/or a Cl alkane. Preferably the Cl compound and/or the Cl alkane may be methane, methanol, natural gas, biogas, syngas or any combination hereof. Even more preferably, the Cl compound and/or the Cl alkane may be methane.

The methanotrophic organisms may preferably be a methanotrophic bacteria, such as Methylococcus Capsulatus.

The methanotrophic bacteria may be provided in a co-fermentation together with one or more heterotrophic bacteria.

The following heterotrophic bacteria may be particularly useful to co-ferment with M. capsulatus; Ralstonia sp.; Bacillus brevis; Brevibacillus agri; Alcaligenes acidovorans; Aneurinibacillus danicus and Bacillus firmus. Suitable yeasts may be selected from species of Saccharomyces and/or Candida.

The preferred heterotrophic bacteria are chosen from Alcaligenes acidovorans (NCIMB 13287), Aneurinibacillus danicus (NCIMB 13288) and Bacillus firmus (NCIMB 13289) and combinations thereof.

In an embodiment of the present invention the methanotrophic organism may be a genetically modified methanotrophic organism and/or the heterotrophic organism may be a genetically modified heterotrophic organism.

The fermentation reactor and/or the fermentation process according to the present invention may have special relevance for the production of single cell protein (SCP) by continuous culture fermentation processes, e.g. by Methylococcus capsulatus.

The preferred methanotrophic bacteria are species of the Methylococcus family, especially Methylococcus capsulatus, which utilize methane or methanol as a carbon source and ammonia, nitrate or molecular nitrogen as a nitrogen source for protein synthesis.

Further details of suitable modifications to the loop reactor and feature on how to run such loop reactor, and processing of resulting biomass may be as described in WO 2010/069313; WO 2000/70014; WO 2003/016460; WO 2018/158319; WO 2018/158322; WO 2018/115042 and WO 2017/080987 which are all incorporated by reference.

A first embodiment the present invention relates to a loop reactor; preferably a U-loop reactor, wherein the loop reactor comprises a loop part. The loop part comprises a downflow part of the loop part, a U-part, an upflow part, and a top tank. The top tank comprises a venting tube for exhausting the gas or gases separated in the headspace of the top tank, also termed the effluent gas. Along the loop part of the fermentation reactor, the loop reactor, members are placed for introducing a gas, e.g. gaseous substrate, like methane, ammonia, atmospheric air, pure oxygen or atmospheric air enriched with pure oxygen into the fermentation reactor. Preferably, the members for introducing gas are placed in the loop part of the loop reactor, even more preferably, the members for introducing gas are placed in the downflow part and/or in the upflow part of the loop reactor. The fermentation reactor (the loop reactor) may be provided with a pump for circulating the fermentation liquid (a circulation pump) in the loop reactor. In one embodiment of the present invention the circulation pump may be installed in the U-part of the fermentation reactor for circulation of the broth in the fermenter. This circulation pump may alternatively, and preferably, be placed in the upper part of the downflow part, e.g. acting as the first pressure controlling device. Throughout the downflow part and/or the upflow part dynamic mixers or a static-mechanical mixing member for dispersion of supplied gases into numerous small fine bubbles into the fermentation liquid. Supply conduits for adding water and nutrient salts, such as phosphate, ammonium, magnesium, calcium, potassium, iron, copper, zinc, manganese, nickel, cobalt and molybdenum in the form of sulphates, chlorides or nitrates, phosphates and pH controlling components may be provided, preferably the supply conduits are provided in the downflow part and/or in the upflow part, preferably the supply conduits are provided in the downflow part. An outlet may be provided in the fermentation reactor (the loop reactor) for draining off fermentation liquid with contents of produced biomass and/or other product substances for downstream processing. The outlet may be placed in the U-part and/or in the top tank. The loop reactor (fermentation reactor) may also be supplied with one or more sensors. Sensors may be provided for sensing or determining the concentrations of the gases and/or ions in question, e.g. CH₄ and O₂, and/or at least one of the ions phosphate, ammonium, nitrate, nitrite, and hydrogen ion (pH), one or more thermo sensors for sensing the temperature of the fermentation liquid in the loop part and/or a foam sensor for sensing if excessive foam has developed in the top tank.

The sensors may deliver signals to a data processing system (PC) (not shown), which may control the entire fermentation process, including the downstream processing equipment.

In order to control foaming and/or turbulence of the fermentation liquid in the top tank to ensure an optimized degassing of effluent gasses and hence, an improved productivity of the fermentation process, the top tank of the loop reactor (the fermentation reactor) may be provided with a visual inspection means. Preferably, the visual inspection means may be placed with a horizontal or substantial horizontal inspection view. The visual inspection means may be placed on the side of the top tank allowing a combined view above the surface of a fermentation liquid and below the surface of the fermentation liquid. Preferably, the visual inspection means may be placed in the end of the top tank. Preferably, the visual inspection means may be placed in the end of the top tank providing a view from the first inlet (or the upflow part) towards the first outlet (or the downflow part). The visual inspection means may be an inspection hole, a camera, or a combination of an inspection hole and a camera. In an embodiment of the present invention the inspection hole may be a sight glass. In another embodiment of the present invention the camera may be an inline camera.

An example of downstream processing suitable for the biomass obtained in order to provide various fraction may be as described in the aforementioned WO 2018/115042.

A second embodiment of the present invention relates to a loop reactor (fermentation reactor) similar to the first embodiment described above in which the pressure may be raised in specific zones of the loop reactor while the pressure is substantially equal to atmospheric pressure, or optionally reduced below atmospheric pressure may be provided in other zones of the loop reactor, e.g. in the top tank.

In order to control the pressure at specific zones of the loop reactor (the fermentation reactor) one or more pressure controlling devices may be placed in the loop reactor to permit the pressure in the fermenter to be controlled, such that different zones of the fermenter experience a higher or lower pressure than other zones, e.g. the loop part of the loop reactor.

In an embodiment of the present invention the devices used to control the pressure are also used to circulate the liquid and/or the gas-liquid mixture in the fermenter, i.e. a circulation pump.

In a preferred embodiment of the invention, a first pressure controlling device may be placed in the top of the downflow part or in a connection parts between the top tank and the downflow part, e.g. in the lower part thereof. The first pressure controlling device may preferably be circulating the liquid in the fermenter, and at the same, time causes an increase in pressure when the liquid or the gas-liquid mixture passes through the first pressure controlling device. The first pressure controlling device may preferably be a pump, e.g. a circulation pump, a propeller pump, a lobe pump or a turbine pump especially designed for circulating a gas-liquid mixture. Other suitable means for increasing the pressure and creating liquid circulation in the fermenter are e.g. addition of a pressurized gas aimed at increasing pressure and not simply for supplying nutrients or ingredients to the fermentation liquid. When the first pressure controlling device may be a pump, such as a propeller pump, the pump, e.g. the propeller pump may be driven by a motor.

A second pressure controlling device may be placed in the loop reactor (fermentation reactor), preferably, in the loop part, e.g. in one of the downflow part or the upflow part or in the horizontal part of the U-part of the loop reactor (fermentation reactor). Preferably, the second pressure controlling device may be placed in the upflow part of the fermentation reactor, such that the pressure may be increased between the first pressure controlling device and the second pressure controlling device when seen in the flow direction.

When the pressure is increased in a specific zone in the fermenter, the solubility of the injected gases in the liquid phase is also increased. In a preferred embodiment, the second pressure controlling device may be placed in the middle to the top of the upflow part of the fermentation reactor (or of the loop part).

The second pressure controlling device may be chosen among a number of pressure controlling devices such as: a narrowing of the diameter/cross section of the upflow part or of the U-part, a plate with holes, jets or nozzles inserted in the upflow part or the U-part, a valve, e.g. a valve controlled by the pressure at one or more locations in the fermenter, a static mixer, a hydro cyclone or a pump, such as a propeller pump, a lobe pump or a turbine pump.

The loop reactor (fermentation reactor) may be provided with one or more pressure sensing devices. The one or more pressure sensing devices may be placed throughout the loop reactor, such as in the top tank, and/or in the loop part.

Preferably, at least one pressure sensor may be placed in each of the zones of the fermenter operated under different pressures. The pressure sensing devices may be connected to a process control system, e.g. a computer, which may control the pressure controlling devices and in order to maintain an optimal pressure in each of the zones of the fermentation reactor.

In respect of both the first and the second embodiment as described above, the loop fermenter (fermentation reactor) according to the present invention may further comprise one or more sensors for determining dissolved oxygen (DO) may also be placed in the fermenter in order to detect if the oxygen level in the fermenter is kept within a predefined range, which depends on the microorganism or microorganisms used in the fermentation.

Additional sensors, e.g. for measuring temperature, pH, conductivity measurements, oxidation reduction potential and different ions present in the broth, e.g. ammonia, nitrite, nitrate, phosphates, etc., may be placed in the fermentation reactor, in the top tank and/or in the U-part.

The sensors may include biosensors, electrochemical sensors, e.g. ion sensitive electrodes or sensors based on FIA (flow injection analysis) and optical measurements, e.g. spectrophotometric devices. A Near Infrared (NIR) probe may also be used for measuring several different components in the broth or in the cells in the fermenter, e.g. concentration of cells, amino acids, methanol, ethanol and/or different ions. The fermentation reactor may also be equipped with a mass spectrometric (MS) sensor or an electronic nose for determining the concentration of gaseous and volatile components (e.g. CO₂ and/or CH₄) in the headspace. The MS sensor or the electronic nose may control the pressure applied in the fermenter and/or the addition of gaseous components, e.g. methane and/or air/oxygen and/or the addition of gaseous ammonia or the ammonia/ammonium in solution. A high-speed camera may be installed in the U-part of the fermentation reactor, preferably in connection with gas injection, for determining the bubble size of the gases in the broth. The bubble size may be determined by image processing of the data from the high-speed camera.

Recirculated supernatant may also be added to the top part of the fermenter, alternatively it may be added at one or more positions in the U-part of the fermentation reactor. Return of supernatant from downstream processing may reduce the overall consumption of substrates, carbon and minerals, thus reducing the costs of the fermentation process.

The connection parts may or may not contain vortex hindering means (not shown), e.g. baffles or the like according to needs.

The fermentation reactor according to the present invention may normally be run in continuous operation mode, after cleaning and a sterilization procedure, followed by a start period in which water, necessary nutrient salts and the microorganisms are added to the fermentation reactor. The fermentation liquid may be circulated in the fermentation reactor, mainly by the first pressure controlling device. Then addition of gaseous substrates may be initiated, and fermentation may be started. When the density of microorganisms has reached a concentration of approximately 0.5-10%, and preferably 1-5% (by dry weight) fermentation liquid may continuously be withdrawn from the fermentation reactor, e.g. from the top tank or from the U-part, and subjected to downstream processing, e.g. as described in the aforementioned WO 2018/115042

Withdrawing of fermentation liquid may be initiated simultaneously with the addition of make-up water, aqueous substrate and/or recirculation of supernatant at a dilution rate depending on the microorganisms used in the fermentation. Addition of substrate components in liquid solution, additional water, recirculation of supernatant as make-up for the withdrawn broth and substrate gases may be controlled by a computer receiving data from the gas sensors and suitable calculations for providing the necessary amounts of each component for obtaining optimized growth of the organisms.

It should be noted that embodiments and features described in the context of one of the aspects of the present invention also apply to the other aspects of the invention.

All patent and non-patent references cited in the present application, are hereby incorporated by reference in their entirety.

### REFERENCES

WO 2010/069313
WO 2000/70014
WO 2003/016460
WO 2018/158319
WO 2018/158322
WO 2018/115042
WO 2017/080987
WO 2018/132379

1. A fermentation reactor comprising a loop-part and a top tank, said loop-part comprising a downflow part, connected to an upflow part via a U-part, wherein the top tank comprises:
(i) a first outlet connecting the top tank to the downflow part of the loop-part and allowing a fermentation liquid present in the top tank to flow from the top tank into the loop-part;
(ii) a first inlet connecting the top tank to the upflow part of the loop-part, allowing fermentation liquid present in the loop-part to flow from the loop part into the top tank; and
(iii) a vent tube for discharging effluent gasses from the top tank;
wherein the top tank further comprises a visual inspection means.

2. The fermentation reactor according to claim 1, wherein the visual inspection means are placed with a horizontal or substantial horizontal inspection view.

3. The fermentation reactor according to claim 1, wherein the visual inspection means is placed on the side of the top tank allowing a combined view above the surface of a fermentation liquid and below the surface of the fermentation liquid.

4. The fermentation reactor according to claim 1, wherein the visual inspection means are placed in the end of the top tank.

5. The fermentation reactor according claim 1, wherein the visual inspection means are placed in the end of the top tank providing a view from the first inlet or towards the first outlet or the downflow part.

6. The fermentation reactor according claim 1, wherein the visual inspection means is an inspection hole, a camera, or a combination of an inspection hole and a camera.

7. The fermentation reactor according to claim 6, wherein the inspection hole is a sight glass.

8. The fermentation reactor according claim 1, wherein the top tank is provided with a foam sensor inside the top tank.

9. The fermentation reactor according claim 1, wherein the fermentation reactor is for the fermentation of methanotrophic organisms.

10. The fermentation reactor according claim 1, wherein the loop-part of the fermentation reactor comprises one or more active devices and/or one or more inactive mixing members for distributing gas in the fermentation liquid
